(19) United States
(12) Patent Application Publication
(10) Pub. No.: US 2016/0001050 A1
(43) Pub. Date: Jan. 7, 2016

(54) URETERAL STENT AND METHOD AND SYSTEM FOR ITS DEPLOYMENT

(71) Applicant: PERCUTANEOUS SYSTEMS, INC., Palo Alto,, CA (US)

(72) Inventors: Steven YEE, Sunnyvale, CA (US); Robert BEHL, Monterey, CA (US); Randy KESTEN, Los Altos, CA (US); Jose GALDOS, San Jose, CA (US)

(21) Appl. No.: 14/766,124

(22) PCT Filed: Jan. 22, 2014
(86) PCT No.: PCT/US2014/012572

§ 371 Date: Aug. 5, 2015

Related U.S. Application Data
(60) Provisional application No. 61/755,109, filed on Jan. 22, 2013.

(51) Int. Cl. A61M 27/00

(52) U.S. Cl. CPC A61M 27/008; A61F 2002/048

(57) ABSTRACT

A ureteral stent includes a shaft having a soft tip and deployable anchor at its distal end and a coiled pigtail at its proximal end. The stent is delivered by a wire assembly which includes an inner wire and a sheath. The inner wire extends through a lumen within the stent shaft and is used to deploy the anchor structure. The wire then separates from the stent so that it may be removed for full stent deployment.

### CROSS-REFERENCE TO RELATED APPLICATIONS

This application claims priority to Provisional Application No. 61/755,109 (Attorney Docket No. 28675-730.101), filed Jan. 22, 2013, the content of which is incorporated herein by reference.

### BACKGROUND OF THE INVENTION

1. Field of the Invention

The present invention relates generally to medical apparatus and methods. More particularly, the present invention relates to methods and ureteral stents for decompressing ureteral stones and blockages in other body lumens.

Calculi or “stones” can form in the kidneys as low solubility waste materials precipitate out of solution. The resulting “kidney stones” can pass into the ureter and, so long as the stones are relatively small, can pass from the patient through normal urination. Larger kidney stones which lodge in the ureter are referred to as “ureteral stones.” Such ureteral stones can cause pain, particularly when a stone occludes the ureter and causes back pressure to build up in the kidneys. Kidney stone incidence (via extended pressurization of the kidney) may also be a precursor to long-term kidney damage and kidney failure, decades later.

Ureteral stones can be treated in a variety of ways. Most commonly, patients are given pain medication and fluids and the stones are allowed naturally pass from the ureter. In the remaining cases, where the pain is more severe or the build-up of pressure threatens to harm the kidney, the patient may be treated more aggressively to remove or destroy the stone. Common treatments include surgery, shock wave lithotripsy, laser lithotripsy, and the like.

Of particular interest to the present invention, stents may be placed from the kidney to the bladder to bypass the ureteral stone and allow drainage and pressure relief. A typical ureteral stent is a 5-7F tube/catheter, with an anchoring coil on either end, one is positioned in the renal pelvis of the kidney, the other in the bladder. Currently, placement of such stents is often after prior treatment. Although it may be desirable to place a stent before treatment to decompress the kidney and relieve both pressure and pain as soon as possible, such prophylactic stent placement is rare. Stent placements are typically performed in an operating room where patient anesthesia is available to ease the manipulation of a relatively large tube past an impacted stone, and fluoroscopic imaging is also available to aid and verify stent positioning.

The need to use an operating room is a disadvantage of present procedure. The risk of traumatizing and/or perforating the ureteral wall as a significant length of stent, is advanced past the stone in order to anchor one end in the kidney is another disadvantage of the present procedures. An additional disadvantage in prophylactic placement of a current stent is that discomfort caused by the stent itself may at least partially offset the relief of symptoms from the decompression. Finally, placement of a current stent typically slows or stops ureteral peristalsis, necessary for spontaneous passage of the stone. “Poisoning” of peristalsis is believed to be positively correlated to the diameter of the stent and negatively to the continuous stimulation of the pacemaker zone for triggering peristalsis within the kidney by the upper coil anchor of the stent. Thus, once a current stent is placed, it is not likely that the stone will progress down the ureter, even with any stent-related ureteral dilation that may occur.

For these reasons, it would be desirable to provide improved ureteral stent designs and methods for their placement. In particular, it would be desirable if the stents had a very low profile for placement, did not require anchoring within the kidney, and could be introduced without the need for full anesthesia and in settings other than an operating room. It would be further desirable if the stents could be deployed with a minimum number of steps, if the stents could accommodate different distances between the stone location and a patient's ureteral os, and in certain circumstances if the stent design would allow the stent to remain within the ureter even if the stone is displaced and anchoring of the stent by the stone in the ureter is reduced or eliminated. At least some of these advantages will be met by the inventions described hereinbelow.

2. Description of the Background Art

U.S. Pat. No. 7,792,292, commonly assigned with the present application and incorporated fully herein by reference, describes a ureteral decompression device comprising a guide member and an anchoring structure. The guide member may be introduced through the urethra and bladder into the ureter so that the anchor structure passes a lodged kidney stone. Once past the stone, but still within the ureter, the anchor structure can be compacted atop the stone, to anchor the guide member in place. The anchor structure will permit leakage and the guide member will provide a leakage path directly past the stone, thus decompressing the kidney. Related U.S. Pat. Nos. 6,214,037 and 6,709,465 and Published Application 2005/00600023 describe ureteral stents comprising a series of adjacent expanding structures for dilating the ureter and capturing/removing a stone, however these devices are anchored in place utilizing the typical kidney and bladder coils and present the same set of problems or more so than the current stents.

### SUMMARY OF THE INVENTION

The present invention provides methods and systems for deploying ureteral stents in a patient's ureter for the purpose of decompressing ureteral stones, more commonly known as kidney stones, which block the patient's ureter and can cause substantial discomfort and pain. By “decompressing,” it is meant that a small leakage path will be created and maintained which bypasses the ureteral stone and allows urine to drain from the kidney to the bladder. Although the volumetric rate of drainage may be small, the ability to drain the kidney at even a very low rate is of great benefit to the patient since it reduces the pain and the risk of damage to the kidneys.

The systems and methods of the present invention are particularly advantageous as they simplify the introduction and placement of ureteral stents and in particular minimize the number of steps necessary to place the stent. As will be described in greater detail below, the guidewire sized ureteral stent incorporates a small internal core wire, where the unified combination can be advanced from an endoscope or other delivery device into the ureter. The stent is first advanced distally and, once the anchor section of the stent is past the ureteral stone, the anchor is first deployed, secondly latched in place, and finally the core wire detached from the stent by simply withdrawing the core wire in a proximal direction out from the stent body and ureter. The stent typically has a pigtail coil positioned in the bladder, which can be uncoiled to a length sufficient to accommodate most if not all stone locations within the ureter and which further is sufficiently resilient to apply a small constant proximal force on the deployed anchor, which force can in many cases help to dislodge the stone and cause the stone to pass from the ureter into the bladder. Further, even when the stone does pass, it is desirable that the stent will remain within the ureter rather than coiling in and potentially irritating the bladder. This is accomplished by making the shaft sufficiently stiff to prevent the stent from backing out of the ureter upon loss of the stone which had acted to hold the stent in place.

In a first aspect of the present invention, a ureteral stent deployment system comprises a ureteral stent with an initially integral core wire, and a pusher tube deployed over the wire similar to the pusher tube of a current stent. Unlike a current stent, the entire assembly of this invention is sized at approximately the size of a guidewire, and the entire assembly has similar handling characteristics to a guidewire. The ureteral stent typically comprises a body having a hollow lumen with a closed distal end and a open proximal end. A deployable anchor is disposed at or near the distal end of the body and is configured to be selectively deployed on a distal (kidney) side of the ureteral stone to anchor the stent in place. This is a particular advantage as it is not necessary to traverse the entire ureter into the kidney, and to then deploy an anchor there, in the region of the peristalsis pacemaker. A smaller diameter and softer tip on the distal tip of the stent body usually extends beyond the stone. The tip is initially stiffened by the core wire so as to facilitate passage beyond an impacted stone, but sufficiently soft that it is still atraumatic. Once the stiffening core wire is removed, the tip becomes exceptionally soft and floppy and will not irritate or otherwise damage the tract over time.

The core wire is removably received in the central lumen of the stent body and extends up into the closed tip of the stent, with the ground down and more flexible distal section of its length. Within the body of the stent, the core-wire is frangibly coupled to the latch portion of the anchor. In this way, the core wire can be used to advance the stent by pushing distally on the wire and pusher tube, while the wire remains coupled to the anchor and the stent. Once the anchor is past the ureteral stone, the physician may pull proximately on the wire, which pulling first deploys the anchor (usually by causing the anchor to fold or accordion) until it is latched in place, and subsequently detaches the core wire from the stent as the frangible coupling is broken. Subsequently, the wire and the pusher tube are withdrawn from the tract, leaving the stent deployed in position. Usually, a pusher tube is slidably mounted over a proximal end of the core wire and butts up to the proximal end of the stent body.

The deployable anchor may have any one of a number of configurations and could, in some instances, be in the form of a cage, a malecot, an expandable braid, or any one of a variety of other structures which are configured to deploy when axially shortened by pulling proximately on the core wire. In an exemplary embodiment, the anchor will be a flat polymeric film which folds or bunches as it is axially shortened. In such cases, the core wire will be frangibly coupled to a distal end of the flat polymeric film or other deployable anchor structure, typically by a short tubular member which is secured to a distal end of the core wire by a frangible coupling. For example, the frangible coupling could be a polymeric sleeve which bridges a distal end of the wire and proximal end of the tube, where the sleeve has a scored or other weakened region which breaks upon sufficient axial tension as the deliver wire is drawn proximally, but not before the anchor is fully deployed and latched. A variety of other frangible couplings, of course, could also be employed.

The ureteral stent will also preferably include a latch or other mechanism which holds the anchor in the deployed configuration even after the core wire has been detached. For example, the latch may comprise an enlarged region or feature disposed on the tubular member which engages a locking region, such as an axially split region, on the stent body as the tubular member is drawn proximally. The axially slit(s) would prove an expansion region to receive and engage the enlarged bump or ring feature on the tube when the anchor was properly deployed. A variety of other latching or locking mechanisms could also be employed.

In a further aspect of the present invention, the ureteral stent comprises a shaft, a soft tip, a pigtail and a deployable anchor located near a junction of the shaft and soft tip. The shaft has a proximal end and a distal end with a lumen extending there between. The soft tip extends distally from the distal end of the shaft and also has a lumen therein, where the soft tip lumen aligns axially with the shaft lumen. The pigtail extends proximally from the proximal end of the shaft and is typically not aligned with the lumens in the shaft and the soft tip. In this way, the core wire may be inserted into the aligned lumens of the shaft and the soft tip for advancement of the stent through the ureter with a temporarily stiffened tip and shaft, while the pigtail remains adjacent to the core wire and fully flexible. While the stent is being deployed through a restraining cystoscope lumen, the soft, unstiffened pigtail will typically uncoil and assume a generally straightened configuration which lies parallel to the axis of the core wire. The pigtail will be maintained in the straightened configuration while it remains constrained within the delivery lumen, such as a the working lumen of an endoscope being used to deploy the ureteral stent as described in more detail below. The core wire is removably insertable into the lumens of both the shaft and the soft tip for delivery of the stent. As described above in connection with the ureteral stent delivery system, the shaft of the ureteral stent will usually be coupled to a distal end of the core wire in such a way that the combined device and pusher tube will have the handling characteristics of a guidewire as is is deployed through the scope and up through the ureter. Once in position, the core wire can be further used to draw proximally on the stent to first deploy the anchor and then further to disengage itself from the stent. Usually, the coil material is softer than the shaft material and the coil force is maintained by an internal spring wire so as to limit irritation resulting from engaging the ureteral Os and bladder wall.

In specific aspects of the present invention, the shaft of the stent will have a stiffness which is greater than, usually significantly greater than that of the soft tip. For example, the shaft may have a bending stiffness in the range from 200 mpa to 400 mpa, preferably from 250 mpa to 350 mpa, and the soft tip will have a bending of stiffness in the range from 20 mpa to 40 mpa, more preferably from 25 mpa to 30 mpa. The shaft usually has a durometer (hardness) in the range from 65 D to 75 D, typically being from 70 D to 72 D, and the soft tip will usually have a durometer (softness) in the range from 75 A to 100 A, usually from 80 A to 90 A.

In another preferred aspect of the present invention, the pigtail coil comprises a coil structure having turns which lie in a plane which is generally parallel to the axis of the stent shaft. The coil will usually have at least two turns, typically having from 2 turns to 4 turns, and can extend to a length of at least 40 mm, typically from 50 mm to 90 mm, when fully uncoiled.

The coil of the pigtail will often be reinforced with a spring wire, such a wire formed from nitinol or other shaped-memory alloy, so that the pigtail coil has a memory which causes the coil to rewind whenever it is unconstrained. In this way, the material utilized to construct the pigtail may be significantly softer than the shaft material, but still have the coil memory imparted by the wire. Further, when the stent is present in the ureter, the partially unwound coil may engage the bladder wall adjacent the ureteral os with the soft outer material, applying a force to the stent which causes the deployed anchor to apply a proximal force on the kidney stone. As discussed in more detail below, the proximal force applied by the partially unwound pigtail coil to the anchor can cause the kidney stone to dislodge and in some instances to be released into the bladder.

In yet another aspect of the present invention, a method for delivering a stent past a stone in a ureter comprises providing a ureteral stent which is removably mounted over a distal end of a core wire. The core wire is pushed distally, typically from its proximal end, to advance the stent through the ureter in a direction toward the kidney and past the stone by applying forward force internally against the anchor and closed distal end of the stent tip. This is aided secondarily by the pusher tube, but the primary advancement comes from the wire, which also simultaneously imparts favorable stiffness and handling characteristics to the tip and stent body. After the distal anchor structure on the stent passes the kidney stone, a physician may draw proximally on the wire while the pushed tube holds the stent in place, to deploy the anchor on the kidney side of the stone. As the physician continues to draw proximally on the core wire, the core wire disengages from the stent, leaving the stent in place in the ureter. The shaft or body of the stent is disposed between the stone and the interior wall of the ureter to provide a leakage path for urine past the stone. The core wire and pusher tube can be removed from the ureter, leaving the ureteral stent in place.

In other aspects of the methods of the present invention, the ureteral stent includes a shaft section proximal to the anchor and a tip section distal to the anchor. The wire extends through aligned lumens present in the shaft, the anchor, and the tip section. Thus, the wire provides stiffness to the stent, and in particular to the distal tip of the stent, as the stent is advanced through the ureter. In further exemplary aspects, the tip is relatively soft to minimize irritation to the ureter and the kidney and the shaft is relatively stiff to limit proximal migration of the stent into the bladder should the anchoring fail, e.g. if the stone were to become dislodged or discharged into the bladder. This occurs as the shaft may be made stiffer than the coil material itself per the above construction, unlike the typical ureteral stent which has both anchors and the body constructed of the same material. The increased body stiffness in a length exceeding 3 cm is too long to allow the stent to back out into the bladder. Exemplary bending stiffnesses for both the tip and the shaft, as well as an exemplary softness range for the tip, have been provided above.

In other aspects of the invention, the stent includes a coiled pigtail at a proximal end of the shaft. This pigtail is typically not disposed over the core wire and is configured to uncoil as the stent shaft is advanced into the ureter, thus allowing a longer pigtail with more coils to be used. As it uncoils, the pigtail engages a wall adjacent the ureteral os in the bladder, thus causing the uncoiling pigtail to exert a proximal force on the shaft. As discussed above, such a proximal force can cause the anchor deployed against the stone to dislodge the stone and at least some cases cause the stone to proceed down the ureter and possibly pass into the bladder.

When deploying the proximal anchor on a distal side of the ureteral stone, a counter force (in a distal direction) is typically applied to the stent by the pusher tube, as the core wire is pulled proximately. The pusher tube is typically a blunt-ended catheter or sleeve of approximately the same diameter of the stent, which is disposed over the core-wire, where the blunt distal end of the catheter or sleeve engages a proximal end of the stent shaft body. After the anchor has been deployed, the sheath or sleeve can be withdrawn together with the core wire.

Should the stent become dislodged or for any other reason backs out of the ureter after deployment, the proximal end of the shaft and/or the pigtail coil will engage a wall of the bladder. As the stent shaft is relatively stiff, the stent will be prevented from fully exiting the ureter as the bladder wall will lock such exiting. The presence of the multiply coiled pigtail on the proximal end of the shaft acts to spread contact over a relatively large surface area. The increased surface area and the softer than typical material of the coils help to reduce trauma or irritation to the bladder wall should the stent dislodge.

### BRIEF DESCRIPTION OF THE DRAWINGS

FIG. 1 is a side view of a ureteral stent constructed in accordance with the principles of the present invention shown in cross-section with portions broken away.

FIG. 2 is a side view of a core wire used for delivering the ureteral stent of FIG. 1, shown with portions broken away.

FIG. 3 is a side view of the ureteral stent of FIG. 1 mounted on the core wire of FIG. 2, shown with portions in cross-section and other portions broken away.

FIG. 4A and 4B illustrate the distal region of the ureteral stent and core wire of FIG. 3, shown with a distal anchor deployed (FIG. 4A) and with the core wire detached from the stent (FIG. 4B).

FIG. 5 illustrates a ureter disposed between a kidney and a bladder and having a kidney stone lodged therein.

FIG. 6A through 6E illustrate use of the ureteral stent and core wire from the present invention for placing the ureteral stent in the ureter past a kidney stone.

### DETAILED DESCRIPTION OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized.

The methods and apparatus of the present invention are useful for decompressing ureteral or kidney stones in the ureter of patient. The ureteral stones can, in some cases, fully occlude a ureter and cause a build-up of pressure within the kidney since urine can no longer pass through the ureter. The decompression devices and methods of the present invention are useful for placing a very small ureteral stent or equivalent structure past the stone in a simpler manner with less steps than are currently utilized, and such that the stent is anchored in place on or in close proximity to a kidney side of the stone rather than the anchor being placed in the renal pelvis of the kidney as occurs with current ureteral stents. The smaller size and the lack of an anchor continuously stimulating the renal nerves which trigger peristalsis will aid in maintaining peristalsis and overcoming the lack of stone passage seen with current stents.

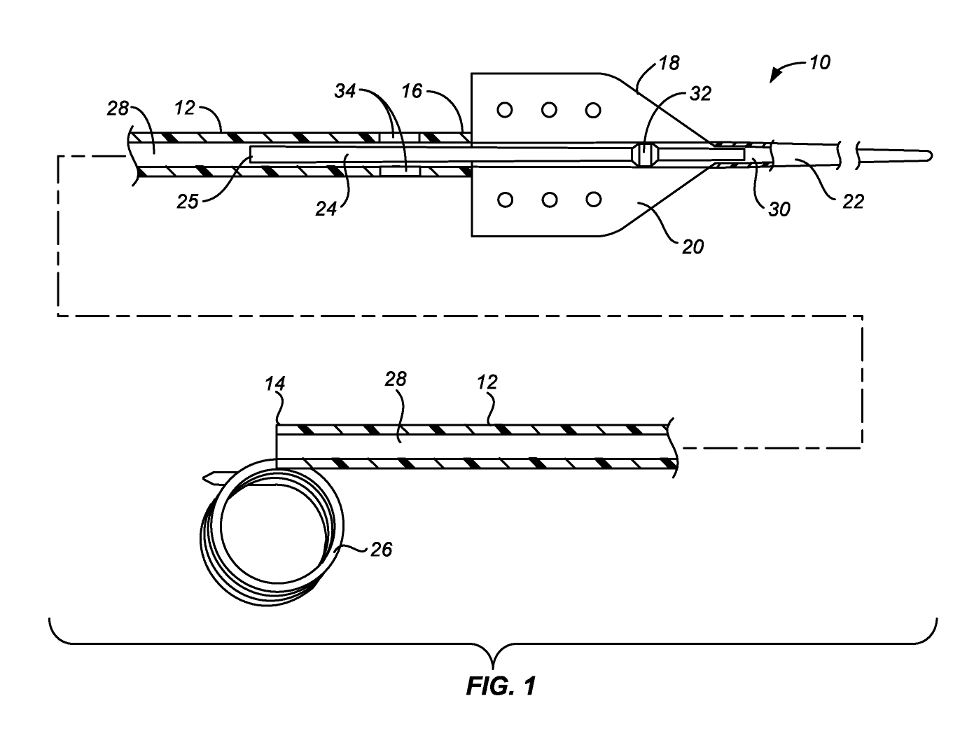

Referring now to FIG. 1, a ureteral stent 10 comprises a shaft 12 having a proximal end 14 and a distal end 16. A deployable anchor structure 18 is located at, and usually attached to, the distal end 16 of the shaft 12. While the deployable anchor structure could take a variety of common forms, such as a malecot, an expandable braid, a wire cage or coil, or some non-occluding inflatable structure, and the like, it will usually be in the form of a thin polymeric film which can be axially folded, bunched or axially compressed to form an enlarged structure which can be positioned on the kidney side of a stone within the ureter to hold the stent in position relative to the kidney stone. The use of film is advantageous as it can be made very thin and slippery to pass the impacted stone, can be made perforated or made porous to allow urine flow, and yet have significant surface area post-deployment to minimize subsequent trauma to the inner ureter as the ureter stretches and contracts. Details on the construction of such polymeric films may be found in commonly owned U.S. Pat. No. 7,879,066, the full disclosure which is incorporated herein by reference.

The ureteral stent 10 further includes a soft tip 22, typically having a closed distal end, which extends from a distal end of the deployable anchor structure 18. The stent shaft 12 will typically be a relatively stiff structure, typically having a bending stiffness as set forth hereinabove. In contrast, the soft tip will be a relatively soft, atraumatic structure, typically having a bending stiffness and a hardness in the ranges set forth above when the inner core wire is removed.

The shaft 12 is joined to the soft tip 22 by the anchor 18 and an interior clutch tube 24 which is also joined to the proximal end of the soft tip. The clutch tube 24 is free to slide within a lumen 28 of the shaft 12. As will be described in more detail below, the clutch tube 24 may be drawn proximally relative to the shaft 12 in order to axially fold or compress the deployable anchor structure 18, causing folding or bunching of the film structure. After the clutch tube 24 is drawn proximally and the anchor structure 18 is deployed, a locking element 32 secured over the clutch tube 24 is captured in a locking receptacle 34 formed in a wall of the shaft 12 near its distal end 16. The locking receptacle could have a variety of configurations, most simply it may be two, three or more slots formed in a wall of the shaft, thus allowing an oversized locking element 32 to become captured by distending the region of the slots.

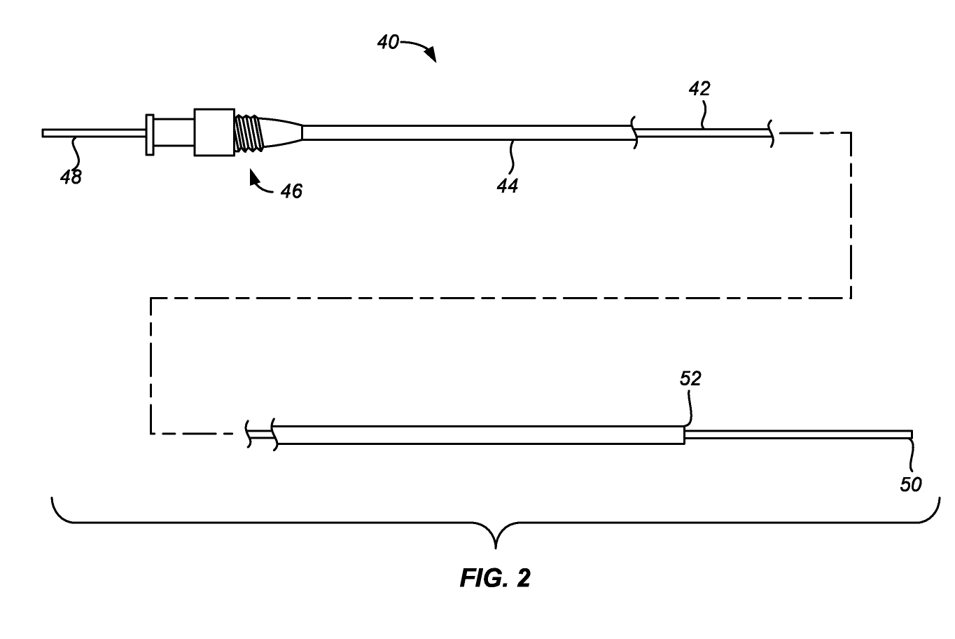

The soft tip 22 will also have a lumen 30 which is aligned with the lumen 28 of the shaft 24. The clutch tube 24 will also be hollow, thus forming a substantially continuous lumen from the proximal end of 14 of the shaft through to the closed distal end of the soft tip 22. As will be described in more detail below, this extended lumen can receive an inner wire 42 of a core wire assembly 40, as shown in FIG. 2.

The ureteral stent 10 further includes a coiled pigtail 26 attached to the proximal end 14 of the shaft 12. The pigtail is attached in such a way that it will not block a proximal opening to the lumen 28 so that the core wire assembly 40 can be introduced therethrough, as will be described in more detail below.

Referring now to FIG. 2, the core wire assembly 40 comprises the inner wire 42 which is co-axially received in a lumen or central passage of a blunt ended pusher tube 44 which is a catheter or sheath which slides over the wire. The pusher tube 44 has a proximal locking hub 46 at its proximal end which allows the inner wire 42 to be selectively locked or immobilized relative to the pusher tube 44. The inner wire 42 includes a handle 48 attached at its proximal end to allow the wire to be advanced and retracted relative to the pusher tube when the locking hub 46 is unlocked. The inner wire 42 has a distal end 50, and the pusher tube has a distal end 52. These distal ends will be used to engage and manipulate the ureteral stent as will be described below.

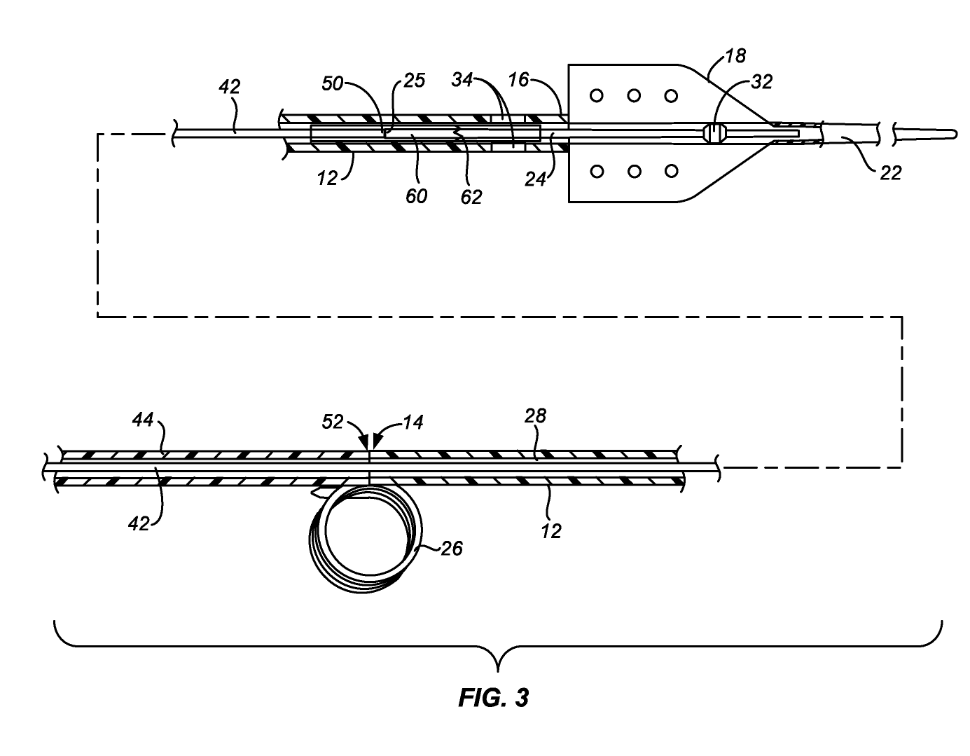

Referring now to FIG. 3, the core wire assembly 40 interfaces with the stent when the core wire 42 is introduced through the proximal opening to lumen 28 in the proximal end 14 of the stent shaft 12 so that distal end 50 of the inner core wire 42 passes through the proximal end 25 of the clutch tube 24 and extends proximally within the interior lumen 30 of the soft tip 22 up to its closed tip, and simultaneously distal end 52 of the pusher tube 44 engages proximal end 14 of the shaft 12. The core wire 42 and the clutch tube 24 are joined by a coupling sleeve 60 which may be a simple polymeric sleeve secured over the proximal portion of the clutch tube 24 and attached to a portion of the core wire 42. The coupling sleeve 60 has a pre-formed tear line 62 which will separate when sufficient tension is applied between the inner wire 42 and the clutch tube 24 as will be described below.

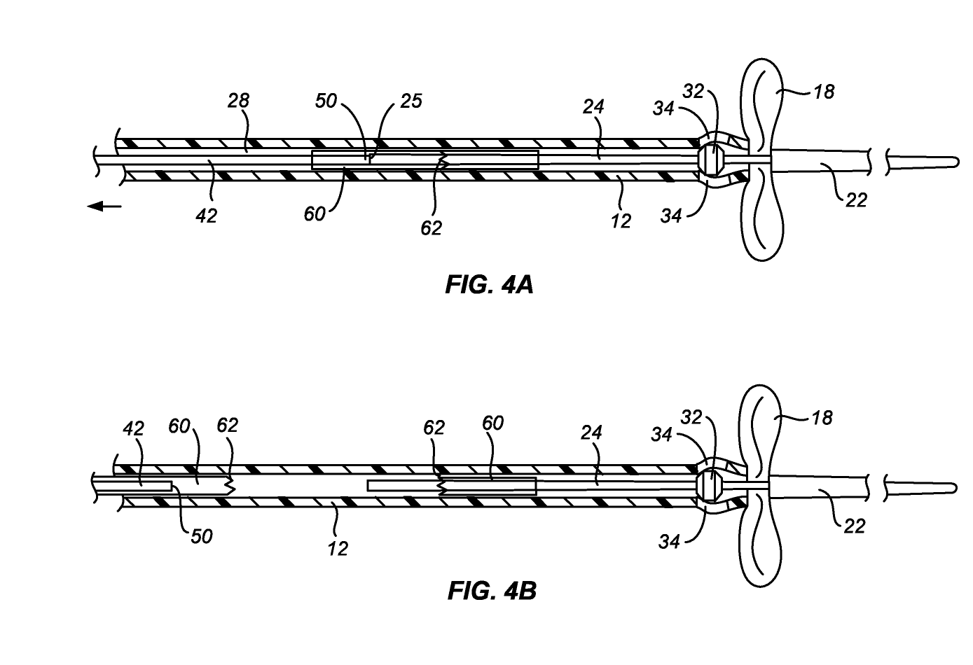

Referring now to FIGS. 4A and 4B, the deployable anchor structure 18 is deployed by pulling proximally on the core wire 42 as the sheath 44 provides counter traction on the shaft 12. A distal portion of the clutch tube 24 is held within the soft tip 22 deploying the anchor 18 into a locked position by pulling locking element 32 into the locking receptacle 34. After the anchor structure 18 is deployed, as shown in FIG. 4A, the proximal pulling force may continue to be applied to the core wire 42 while maintaining counter traction with the sheath 44 until the applied force is sufficient to separate the tear line 62 on coupling sleeve 60, as shown in FIG. 4B. This allows the core wire 42 to be completely removed from the ureteral stent. Once the wire is removed, the sheath 44 and remainder of the core wire assembly 40 may also be removed, leaving the stent in place. The enlarged locking element 32 on the clutch tube 24 which is received in the locking receptacle 34 on the shaft 12 will maintain the stent anchor 18 in the deployed configuration of FIG. 4B after the core wire assembly 40 is removed from the body.

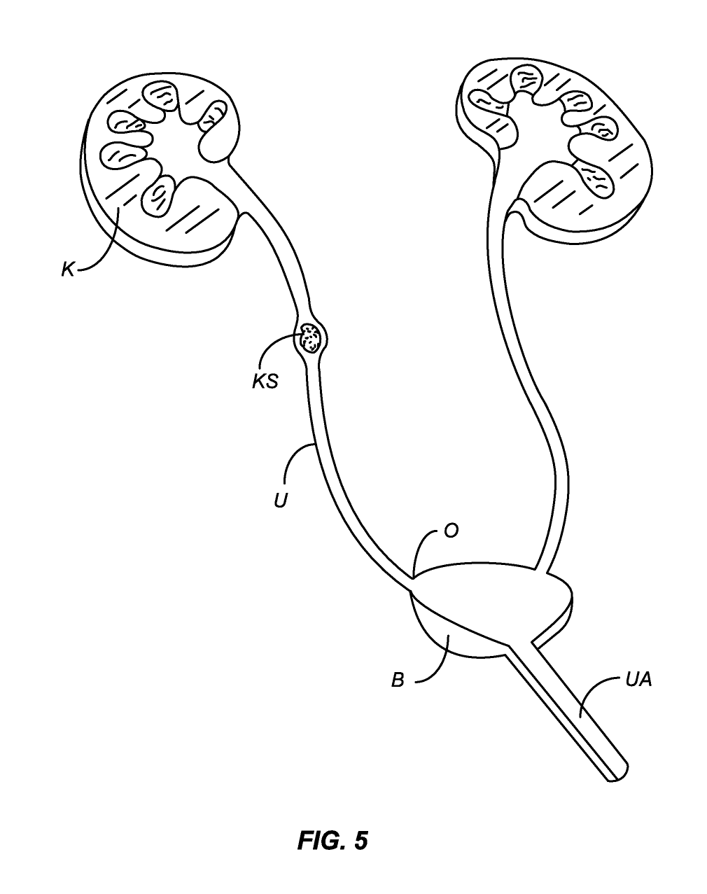

Referring now to FIG. 5, the patient's urinary system includes a ureter U having a kidney K at a distal (superior) end and a bladder B at a proximal (interior) end. The bladder B is connected to the interior of the ureter at an os or ureteral orifice 0. Urine produced in the kidney K flows through the ureter into the bladder B and is eventually released through the urethra UA. The presence of a kidney stone KS in the ureter can limit the flow of urine from the kidney into the bladder, causing pressurization of the upper tract, and pain as discussed previously.

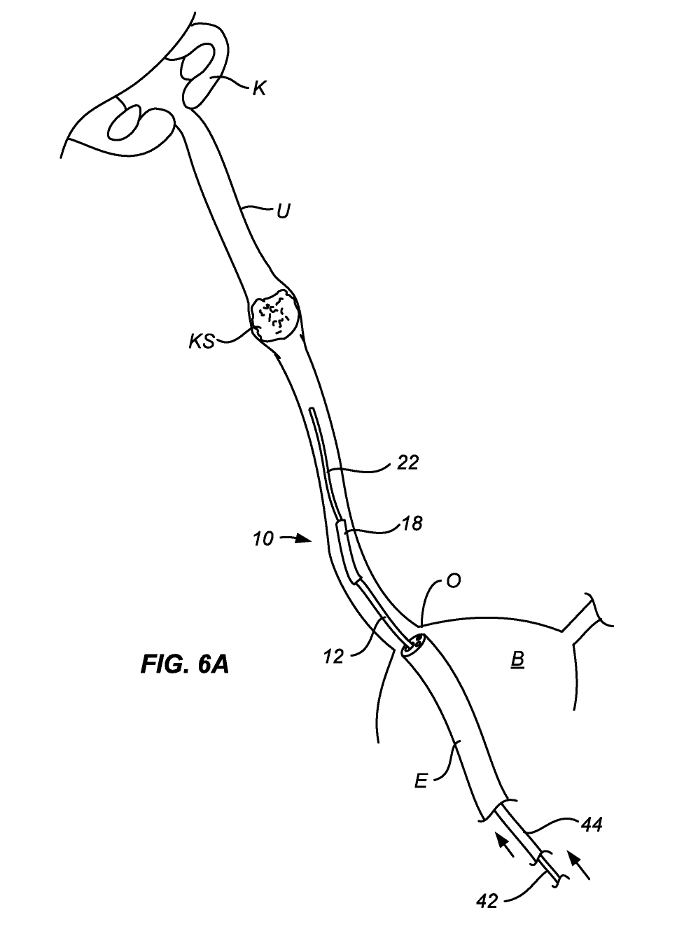

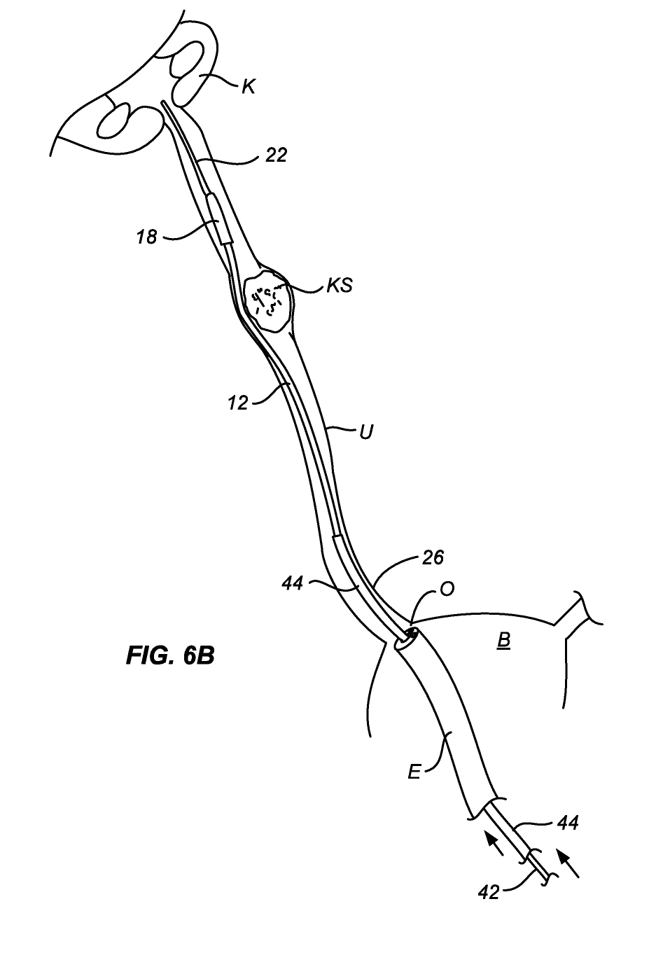

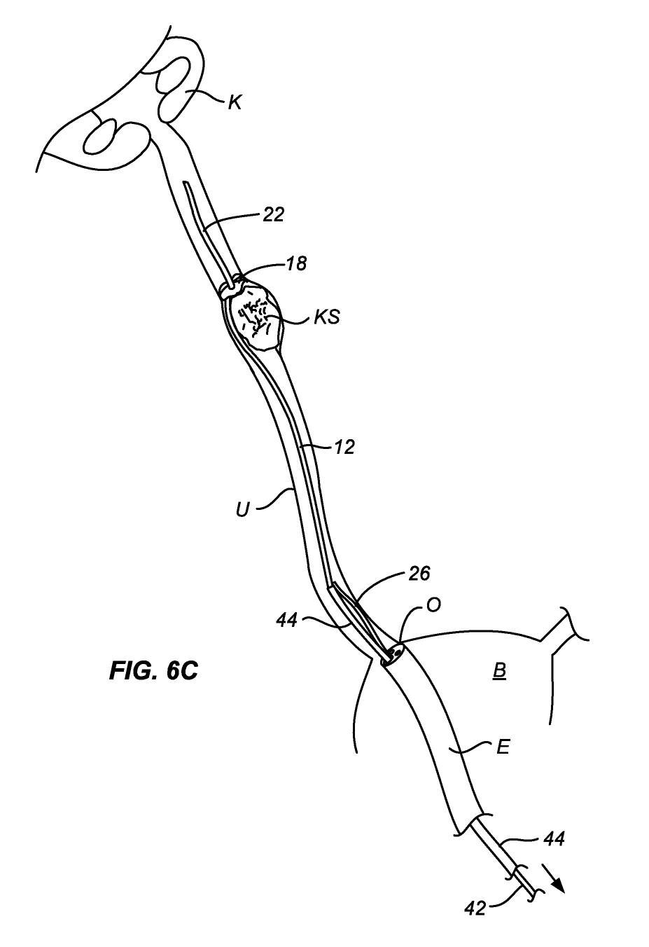

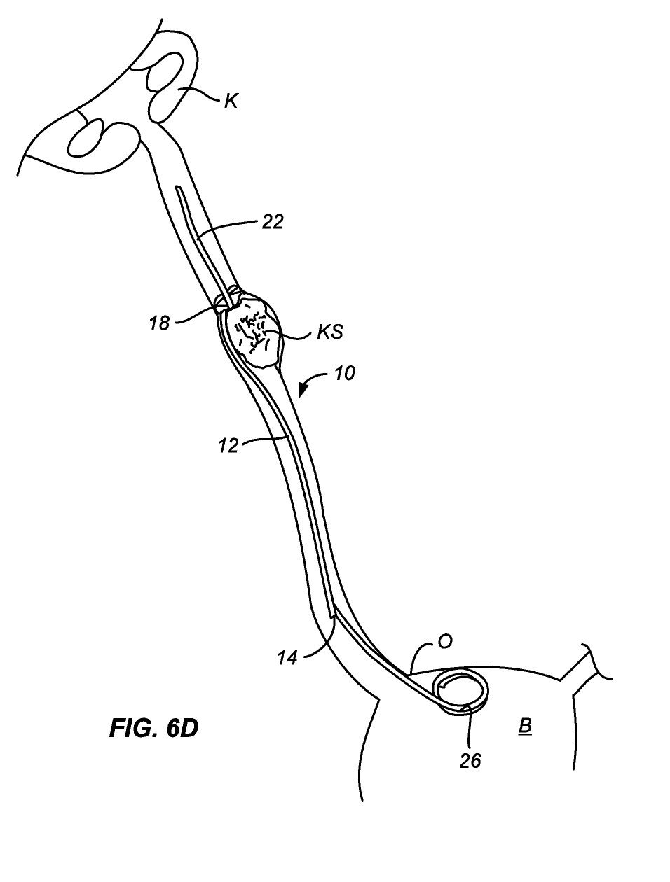

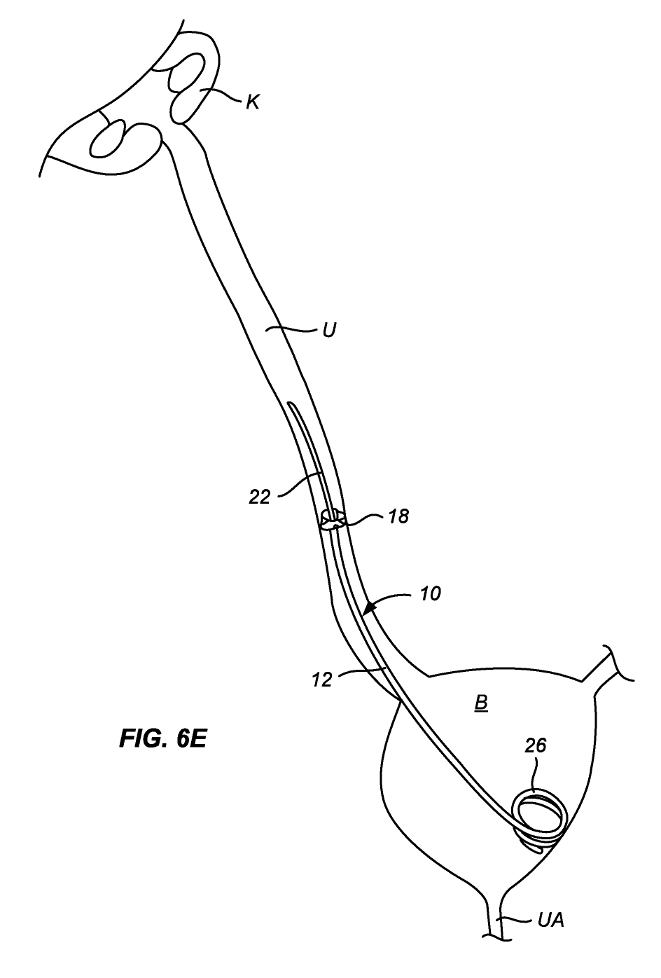

Referring now to FIG. 6A through 6E, the ureteral stent 10 of the present invention may be introduced into the ureter U through an endoscope E which is advanced to the bladder by conventional means. The stent 10 is mounted on the core wire assembly 40 and introduced through a working channel of the endoscope E so that it passes through the os 0 with the soft but temporarily stiffened tip entering the ureter. The ureteral stent 10 is advanced by the physician pushing on a proximal end the delivery assembly so that the soft tip 22 and deployable anchor 18 pass the kidney stone KS, as shown in FIG. 6B. During this advancement, the coil of pigtail 26 is uncoiled as it is constrained in the working channel of the endoscope E.

After the deployable anchor structure 18 has passed the kidney stone KS, the anchor may be deployed by pulling proximally on the core wire 42 while the sheath 44 is held to apply counter traction, as shown in FIG. 6C. The core wire assembly 40 is then pulled proximally leaving the deployed anchor structure 18 above or engaging the kidney side of the kidney stone KS, as also shown in FIG. 6C.

After the deployable anchor has been deployed, the core wire 42 is pulled with a stronger force, while maintaining counter traction with the sheath 44, so that the coupling sleeve 60 separates as shown in FIG. 4B, thus allowing the core wire assembly 40 to be removed through the endoscope such that the ureteral stent 10 is fully deployed, as shown in FIG. 6D. In this deployed configuration, the partially uncoiled pigtail 26 has a tendency to recoil and thus applies a gentle force against the wall of the bladder adjacent the os O. The soft material of the coil allowed by the coil memory being imparted by an interior nitinol wire provides less irritation than does a typical coil formed from the relatively stiff shaft material. This gentle force, however, over time will often cause the deployed anchor 18 to pull downwardly on the kidney stone KS, particularly in conjunction with ureteral stretching due to changes in body position, potentially dislodging the kidney stone and in many instances facilitating kidney stone towards or into the bladder. Should the kidney stone pass into the bladder or become significantly dislodged, the anchoring force provided by deployed anchor 18 diminishes and the stent 10 will have a tendency to back out of the ureter, as shown in FIG. 6E. The relatively stiff shaft 12 of the stent, however, will usually maintain the stent within the ureter when the coiled pigtail 26 engages the opposed bladder wall while serving as a shock absorber.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A ureteral stent deployment system comprising:
a stent having a body with a central lumen, a distal end, a proximal end, and a deployable anchor on the distal end; and
a core wire, wherein the core wire is removably received in the central lumen and frangibly coupled to the anchor, wherein pushing the core wire in a distal direction advances the stent through a ureter so that the anchor passes a stone and pulling the core wire in a proximal direction first deploys the anchor on a kidney side of the stone and then detaches the core wire from the stent leaving the stent in place.

2. A ureteral stent deployment system as in claim 1, wherein the deployable anchor comprises a structure which is configured to deploy by axial shortening.

3. A ureteral stent deployment system as in claim 1, wherein the structure is a flat polymeric film which folds or bunches as it axially shortens.

4. A ureteral stent deployment system as in claim 2, wherein the core wire is frangibly coupled to a distal end of the structure so that pulling the wire proximally shortens and deploys the anchor prior to the wire detaching from the anchor.

5. A ureteral stent deployment system as in claim 4, further comprising a frangible coupling which consists of a scored polymer sleeve.

6. A ureteral stent deployment system as in claim 4, wherein the structure further comprises a latch which engages the body of the stent to hold the anchor in a deployed configuration after the wire is detached.

7. A ureteral stent deployment system as in claim 1, wherein the body comprises:
a shaft having a lumen extending from a distal end to a proximal end thereof;
a soft tip extending distally from the distal end of the shaft and having a lumen axially aligned with the shaft lumen, wherein the central passage in the body is defined by the shaft and tip lumens;
a pigtail extending proximally from the proximal end of the shaft; and
a deployable anchor located near a junction of the shaft and the soft tip;
wherein the core wire is removably insertable in the shaft and soft tip lumens.

8. A ureteral stent deployment system as in claim 7, wherein the shaft has stiffness which is greater than that of soft tip.

9. A ureteral stent deployment system as in claim 8, wherein the shaft has a bending of stiffness in the range from 200 mpa to 400 mpa and the soft tip has a bending stiffness in the range from 20 mpa to 40 mpa.

10. A ureteral stent deployment system as in claim 7, wherein the pigtail is not disposed over the core wire.

11. A ureteral stent deployment system as in claim 7, wherein the pigtail is a coil having at least two turns which can extend to a length of at least 50 mm.

12. A ureteral stent deployment system as in claim 7, wherein the coil is reinforced with a spring wire.

13. A ureteral stent deployment system as in claim 12, wherein the spring wire comprises a superelastic material.

14. A ureteral stent deliverable via a removable core wire for placement in a ureter past a stone, said stent comprising:
a shaft having a lumen extending from a distal end to a proximal end thereof;
a soft tip extending distally from the distal end of the shaft and having a lumen axially aligned with the shaft lumen;
a pigtail extending proximally from the proximal end of the shaft; and
a deployable anchor located near a junction of the shaft and the soft tip;
wherein the core wire is removably insertable into the shaft and soft tip lumens.

15. A ureteral stent as in claim 14, wherein the lumen of the soft tip is sealed at the distal end so as to receive the tip of an internal wire.

16. A ureteral stent as in claim 14, wherein the shaft has stiffness which is greater than that of soft tip.

17. A ureteral stent as in claim 16, wherein the shaft has a bending stiffness in the range of 200 mpa to 400 mpa and the soft tip has a bending stiffness in the range from 20 mpa to 40 mpa.

18. A ureteral stent as in claim 14, wherein the pigtail is a coil having at least two turns which can extend to a length of at least 50 mm.

19. A ureteral stent as in claim 18, wherein the coil is reinforced with a spring wire.

20. A ureteral stent as in claim 19, wherein the spring wire comprises a superelastic metal.

21. A method for delivering a stent past a stone in a ureter, said method comprising:
providing a stent removably mounted over a distal end of a core wire;
pushing distally on the core wire to advance the stent toward a kidney past the stone;
drawing proximally on the core wire to deploy an anchor on the stent on a kidney side of the stone;
continuing to draw proximally on the core wire to disengage the core wire from the stent, leaving the stent in place in the ureter creating a leakage path past the stone; and
removing the core wire from the ureter.

22. A method as in claim 21, wherein the stent includes a shaft section proximal of the anchor and a tip section distal to the anchor, wherein the core-delivery wire extends through a lumen in the shaft, through the anchor, and into a lumen of the soft tip, wherein the delivery wire provides additional stiffness to the shaft and to the soft tip as the stent is advanced.

23. A method as in claim 22, wherein the tip is relatively soft to minimize irritation to the ureter and kidney and wherein the shaft is relatively stiff to limit proximal migration into a bladder.

24. A method as in claim 22, wherein the tip has a durometer in the range from 75 A to 100 A and the shaft has a durometer in the range from 65 D to 75 D.

25. A method as in claim 23, wherein the shaft has a bending stiffness in the range from 300 mpa to 400 mpa.

26. A method as in claim 21, wherein the stent further includes a coiled pigtail on a proximal end of the shaft, wherein the pigtail is not disposed over the core wire, uncoils as the stent shaft is advanced into the ureter and engages a ureteral os, and recoils upon entering the bladder.

27. A method as in claim 25, wherein the coiled pigtail remains partially unrolled after the anchor is deployed after the core wire is removed, wherein the partially unrolled coiled pigtail exerts a proximal force on the shaft to draw the anchor against the stone.

28. A method as in claims 26, wherein the coiled pigtail material is softer than the shaft material and the proximal force is maintained by an internal spring wire so as to limit irritation in engaging the ureteral os and bladder wall.

29. A method as in claim 21, wherein drawing proximally on the core wire includes applying a counter force to the anchor to cause the anchor to foreshorten and expand radially.

30. A method as in claim 29, wherein applying a counter force includes holding a proximal end of a catheter or sheath disposed over the delivery wire, wherein a distal end of the sheath engages the proximal end of the stent body as the delivery wire is drawn proximally.

31. A method as in claim 21, wherein a proximal end of the coiled pigtail in the bladder, engages an opposing wall of the bladder to maintain the stiffer distal end of the shaft in the ureter if the stent has displaced proximally in the ureter over time.

32. A method as in claim 21, wherein the anchor comprises a thin polymer film which compacts or folds into a structure which is large enough to resist being inadvertently drawn proximally past an impacted ureteral stone when the core-delivery wire is drawn proximally.

33. A method as in claim 32 wherein a distal end of the core-delivery wire is frangibly connected to a distal end of the polymer film, wherein the frangible connection breaks after the film is folded or compacted and latched into position.
